(19) United States
(12) Patent Application Publication
(10) Pub. No.: US 2019/0110994 A1
(43) Pub. Date: Apr. 18, 2019

(54) PHARMACEUTICAL COMPOSITION OF DAPAGLIFLOZIN

(71) Applicant: LUPIN LIMITED, Mumbai, Maharashtra (IN)

(72) Inventors: Chandrakant SHERVI, Pune, Maharashtra (IN); Byomakesh PANDA, Pune, Maharashtra (IN); Murali Krishna BHAVARISETTI, Pune, Maharashtra (IN); Subhasis DAS, Pune, Maharashtra (IN); Vijaya Kumar THOMMANDRU, Pune, Maharashtra (IN)

(73) Assignee: LUPIN LIMITED, Mumbai, Maharashtra (IN)

(21) Appl. No.: 16/089,898

(22) PCT Filed: Mar. 30, 2017
(86) PCT No.: PCT/IB2017/051823

§ 371 Date: Sep. 28, 2018

(30) Foreign Application Priority Data
Mar. 31, 2016 (IN) 201621011351

(51) Int. Cl. A61K 9/20; A61K 31/7034; A61K 9/28

(52) U.S. Cl. CPC A61K 9/2018; A61K 31/7034; A61K 9/2027; A61K 9/28; A61K 9/2059; A61K 9/2095; A61K 9/2054

(57) ABSTRACT

Present invention discloses the stable pharmaceutical composition comprising premix of dapagliflozin with at least one pharmaceutically acceptable excipient(s) and process for preparation thereof. Dapagliflozin is highly hygroscopic and hence it is difficult to formulate dapagliflozin as stable pharmaceutical composition. Present invention discloses the stable pharmaceutical composition of dapagliflozin comprising premix of dapagliflozin with lactose.

### FIELD OF THE INVENTION

The present invention relates to solid oral pharmaceutical compositions comprising premix of dapagliflozin with at least one pharmaceutically acceptable excipient(s) and process for preparation thereof.

### BACKGROUND OF INVENTION

Diabetes is a well-known metabolic disease characterized by improper control of sugar in the blood because the body does not produce enough insulin, or properly use insulin, to maintain safe blood sugar levels. High blood sugar levels lead to many complications including blindness, stroke, and nerve damage, amputation of the lower limbs, kidney failure, and heart attack. The incidence of this disease is growing fast. Each year more than 4 million people die from complications of diabetes including heart diseases, strokes & kidney failure.

Sodium glucose cotransporter-2 (SGLT-2) has been discovered to be a new target for treating diabetes in recent years. SGLT-2 is mainly distributed in renal proximal tubules. It was responsible for at least 90% of the glucose reabsorption in the kidney.

Dapagliflozin is an inhibitor of sodium dependent glucose transporter which is chemically represented as (1S)-1,5-anhydro-1-C-{4-chloro-3-[(4-ethoxyphenyl)methyl] phenyl}-D-glucitol having structural formula as represented by formula (I)

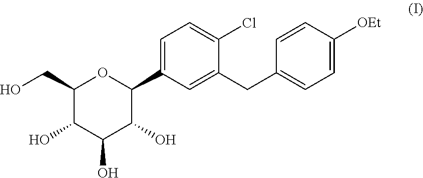 [SMILES: CCOc1ccc(Cc2cc([C@@H]3O[C@H](CO)[C@@H](O)[C@H](O)[C@H]3O)ccc2Cl)cc1]

Currently, Dapagliflozin is approved under the brand name Farxiga Eq 5 mg and 10 mg in the form of tablets, which is marketed by AstraZeneca. U.S. Pat. No. 6,515,117 discloses dapagliflozin as a compound. U.S. Pat. No. 7,919,598 discloses the (S)-propylene glycol solvate of dapagliflozin and processes of preparation thereof. The commercially available formulations of dapagliflozin contain the Propanediol (propylene glycol) monohydrate solvate of dapagliflozin as the active ingredient.

WO 2008/002824 discloses crystalline forms of Dapagliflozin processes for preparing same, intermediates used in preparing same, and methods of treating diseases such as diabetes using such structures.

WO 2012/163546 discloses pharmaceutical compositions comprising cyclodextrin and dapagliflozin, preferably as an inclusion complex.

WO 2014/178040 discloses novel co-crystal forms of dapagliflozin, namely a dapagliflozin lactose co-crystal and a dapagliflozin asparagine co-crystal, to pharmaceutical compositions comprising same, methods for their preparation and uses thereof for treating type 2 diabetes.

The dapagliflozin base is hygroscopic in nature. It absorbs moisture and forms sticky lumps which are difficult to process and handle, and which may ultimately lead to stability and processing problems during manufacturing.

There is therefore an existing and continual need for stable and therapeutically equivalent oral solid pharmaceutical compositions of Dapagliflozin. The compositions of the present invention overcome all the encountered problems exemplified above.

### SUMMARY OF THE INVENTION

The present invention relates to pharmaceutical compositions comprising premix of dapagliflozin with at least one pharmaceutically acceptable excipient(s).

### OBJECTIVES OF INVENTION

Broadly, the object of the invention is to provide solid oral pharmaceutical compositions comprising of dapagliflozin premix with at least one pharmaceutically acceptable excipient(s). The invention also relates to methods of making a pharmaceutical composition comprising dapagliflozin premix.

Another object of the invention is to provide the pharmaceutical composition comprising of dapagliflozin premix for use in the treatment or delaying the progression or onset of diabetes.

In yet another object of the invention is to provide solid oral pharmaceutical compositions comprising of dapagliflozin premix prepared by different methods like dry granulation, wet granulation, direct compression and other suitable methods known to the persons skilled in the art.

In another object, the present invention further provides pharmaceutical composition comprising dapagliflozin premix in combination therapy with one or more other active ingredients in a single pharmaceutical composition or separate pharmaceutical composition.

In another object, the present invention further provides pharmaceutical composition comprising premix of dapagliflozin with lactose in combination therapy with one or more other active ingredients in a single pharmaceutical composition or separate pharmaceutical composition.

In yet another aspect, the one or more active ingredients which optionally employed in combination therapy may include, but are not limited to other type of antidiabetic agents and/or other types of therapeutic agents.

In another object of the invention is a solid pharmaceutical composition which is bioequivalent to the marketed composition of dapagliflozin tablets.

### DETAILED DESCRIPTION OF THE INVENTION

The present invention relates to solid oral pharmaceutical compositions of dapagliflozin premix with at least one pharmaceutically acceptable excipient(s) and process for preparation thereof. More particularly, pharmaceutical composition is in the form of tablet.

“Dapagliflozin” as depicted in formula I used in the present invention is in the form of premix.

In one embodiment, Dapagliflozin premix is a premix of dapagliflozin with lactose. The process for the preparation of the premix of dapagliflozin with lactose comprising the steps of:

(a) providing solution of dapagliflozin;
(b) preparing D-P complex of dapagliflozin of formula (II)

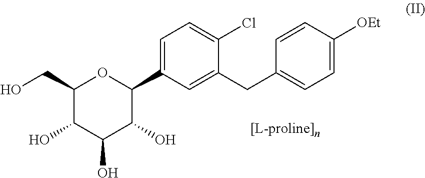

wherein n is 3 to 15;
(c) converting D-P complex of dapagliflozin of formula (II) into dapagliflozin;
(d) providing solution of dapagliflozin obtained in step (c);
(e) precipitating dapagliflozin by treating the solution of step (d) with an antisolvent;
(f) adding lactose;
(g) isolating premix of dapagliflozin with lactose.

In another embodiment, the weight ratio of dapagliflozin to the lactose is from about 1:0.01 to 1:100, preferably 1:0.1 to 1:10.

In one aspect of the present invention, Solid oral pharmaceutical compositions of dapagliflozin premix can be formulated in different oral dosage forms. Such as, but are not limited to powders, granules, pellets, tablets (single layered tablets, multilayered tablets, mini tablets, bioadhesive tablets, caplets, matrix tablets, tablet within a tablet, mucoadhesive tablets, immediate release tablets, sustained release tablet, extended release tablet, modified release tablets, pulsatile release tablets, and timed release tablets), beads, granules, sustained release formulations, capsules, microcapsules, tablets in capsules, microspheres, matrix formulations, microencapsulation, or capsules.

In another aspect, the composition of the present invention can be uncoated or coated form.

In another aspect, pharmaceutical composition of present inventions can be used for the treatment or prevention of diabetes.

In one embodiment, a solid oral pharmaceutical composition is in the form of tablet comprising Dapagliflozin premix and one or more pharmaceutically acceptable excipient(s) prepared by wet granulation method.

In another embodiment, a solid oral pharmaceutical composition is in the form of tablet comprising Dapagliflozin premix and one or more pharmaceutically acceptable excipient(s) prepared by dry granulation method.

In yet another embodiment, a solid oral pharmaceutical composition is in the form of tablet comprising Dapagliflozin premix and one or more pharmaceutically acceptable excipient(s) prepared by direct compression method.

In another aspect, the present invention further provides pharmaceutical composition comprising dapagliflozin premix in combination therapy with one or more other active ingredients in a single pharmaceutical composition or separate pharmaceutical composition.

In another aspect, the present invention further provides pharmaceutical composition comprising premix of dapagliflozin with lactose in combination therapy with one or more other active ingredients in a single pharmaceutical composition or separate pharmaceutical composition.

In yet another aspect, the one or more active ingredients which optionally employed in combination therapy may include, but are not limited to other type of antidiabetic agents and/or other types of therapeutic agents.

The other type of antidiabetic agent which optionally employed in combination may include, but are not limited to one or more antidiabetic agents or antihyperglycemic agents including insulin secretagogues or insulin sensitizers, or other antidiabetic agents preferably having a mechanism of action different from SGLT2 inhibition and may include biguanides, sulfonyl ureas, glucosidase inhibitors, PPAR γ agonists such as thiazolidinediones, aP2 inhibitors, PPAR α/γ dual agonists, dipeptidyl peptidase IV (DPP4) inhibitors, and/or meglitinides, as well as insulin, glucagon-like peptide-1 (GLP-1), PTP1B inhibitors, glycogen phosphorylase inhibitors and/or glucos-6-phosphatase inhibitors.

The other types of therapeutic agents which are optionally employed in combination may include, but are not limited to anti-obesity agents, antihypertensive agents, antiplatelet agents, antiatherosclerotic agents and/or lipid lowering agents.

The term ‘pharmaceutically acceptable excipient(s)’ used in the pharmaceutical compositions of invention comprise but are not limited to diluents, binders, disintegrants, glidants, lubricants, stabilizers, surfactants, solubility enhancers, coloring agents, flavouring agents, sweetening agents.

The amount of excipient(s) employed will depend upon how much active agent is to be used. One excipient(s) can perform more than one function.

Suitable diluents as used in the present invention comprises but are not limited to lactose, microcrystalline cellulose, starch, calcium phosphate, dextrin, dextrose, dextrates, mannitol, sorbitol, sucrose, and the like. Preferably, the diluents are lactose, starch and microcrystalline cellulose.

Suitable binders as used in the present invention comprises but are not limited to, starches such as potato starch, wheat starch, corn starch; microcrystalline cellulose such as products known under the registered trademarks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Suitable disintegrants as used in the present invention comprises but are not limited to, alginic acid, calcium phosphate, tribasic, carboxymethylcellulose calcium, carboxymethylcellulose sodium, colloidal silicon dioxide, croscarmellose sodium, crospovidone, docusate sodium, guar gum, low substituted hydroxypropyl cellulose, magnesium aluminum silicate, methylcellulose, microcrystalline cellulose, povidone, sodium alginate, sodium starch glycolate, polacrilin potassium, silicified microcrystalline cellulose, starch or pre-gelatinized starch or mixtures thereof.

Suitable lubricants as used in the invention comprises but not limited to magnesium stearate, calcium stearate, glycerine monostearate, glyceryl behenate, glyceryl palmitostearate, hydrogenated castor oil, hydrogenated vegetable oil type I, magnesium lauryl sulphate, medium-chain triglycerides, poloxamer, polyethylene glycol, sodium benzoate, sodium chloride, sodium lauryl sulphate, sodium stearyl fumarate, stearic acid, talc, sucrose stearate and zinc stearate.

Suitable Glidants as used in the invention comprises but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

Suitable stabilizers as used in the invention comprises but are not limited to, sodium bicarbonate, ammonium carbonate, anhydrous sodium carbonate, sodium carbonate monohydrate, sodium tartrate, sodium potassium tartrate, sodium citrate, sodium hydroxide, calcium acetate, sodium acetate, dibasic sodium phosphate, anhydrous dibasic sodium phosphate, diammonium hydrogen phosphate, calcium leavinulate, sodium pyrophosphate, and mixtures thereof.

Suitable surfactants as used in the invention comprises but are not limited to, sodium lauryl sulfate, sodium dodecyl sulfate, ammonium lauryl sulfate, benzalkonium chloride, alkyl poly(ethylene oxide), copolymers of poly(ethylene oxide) and poly(propylene oxide) commercially called as poloxamers or poloxamines, polyvinyl alcohol, fatty alcohols, polyoxyethylene alkyl ether, polyoxyethylene alkylaryl ether, polyethylene glycol fatty acid ester, alkylene glycol fatty acid mono ester, sucrose fatty acid ester, sorbitan fatty acid mono ester, sorbitol mono laurate, polyoxyethylene sorbitan fatty acid ester (polysorbates), and mixtures thereof.

Suitable solubility enhancers as used in the invention comprises but are not limited to, dimethylisosorbide, polyethylene glycol, propylene glycol, glycerol, sorbitol sodium lauryl sulfate, glycerol monostearate, glycerol behenate, triglycerides, mono-alcohols, higher alcohols, dimethylsulfoxide, dimethylformamide, N,N-dimethylacetamide, N-methyl-2-pyrrolidone, N-(2-hydroxyethyl) pyrrolidone, 2-pyrrolidone, and mixtures thereof.

Suitable sweetening agents as used in the invention comprises but are not limited to, gluconate, aspartame, cyclamate, sodium saccharine, xylitol and maltitol, or mixtures thereof.

Suitable flavoring agents, coloring agents are selected from any FDA approved flavors, colorants for oral use.

The pharmaceutical compositions disclosed herein can further comprise antioxidants and chelating agents. For example, the pharmaceutical compositions can comprise butylated hydroxyanisole (BHA), butylated hydroxytoluene (BHT), propyl gallate (PG), sodium metabisulfite, ascorbyl palmitate, potassium metabisulfite, disodium EDTA (ethylenediamine tetraacetic acid; also known as disodium edetate), EDTA, tartaric acid, citric acid, citric acid monohydrate, and sodium sulfite.

Suitable Coating agents as used in the invention comprises but are not limited to, cellulose derivatives, e.g., methyl cellulose, hydroxymethyl cellulose, hydroxyethyl cellulose, hydroxypropyl cellulose, hydroxymethylethyl cellulose, hydroxypropylmethyl cellulose, sodium carboxymethyl cellulose, and ethyl cellulose; vinyl polymers, e.g., polyvinylpyrrolidones; acrylic polymers; and mixtures thereof. Alternatively, commercially available coating compositions comprising film-forming polymers marketed under various trade names, e.g. Opadry®, may be used for coating.

The coating additives comprise one or more of plasticizers, glidants or flow regulators, lubricants, coloring agents, and opacifiers. Suitable plasticizers are selected from the group comprising castor oil, diacetylated monoglycerides, dibutyl sebacate, diethyl phthalate, glycerin, polyethylene glycols, propylene glycols, triacetin, triethyl citrate, and mixtures thereof. An opacifier such as titanium dioxide may also be present in the coating.

Suitable solvents as used in the invention for preparing the coating solution comprises but are not limited to water, methyl alcohol, ethyl alcohol, isopropyl alcohol, n-butyl alcohol, acetone, acetonitrile, chloroform, methylene chloride, and mixtures thereof. All these excipient(s) can be used at levels well known to the persons skilled in the art.

The following examples are provided to describe the invention in further detail. These examples, which set forth the best mode presently contemplated for carrying out the invention, are intended to illustrate and not to limit the invention.

### Examples

### Example 1

| Ingredients | % w/w |
|---|---|
| Dapagliflozin Premix | 3.88 |
| Lactose | 48.54 |
| Microcrystalline Cellulose | 23.30 |
| Pregelatinized starch | 11.65 |
| Crospovidone | 7.77 |
| Colloidal Silicon Dioxide | 1.17 |
| Magnesium Stearate | 0.78 |

Film coating

| | |
|---|---|
| Opadry coating | 2.91 |

### Procedure:

1. Sift Dapagliflozin premix and Lactose.
2. Co-sift microcrystalline cellulose, Pregelatinized Starch, Crospovidone and Colloidal silicon Dioxide.
3. Load material of Step 1 & 2 into Blender and blend.
4. Lubricate the blend of step 3 and compress into tablets.
5. Coat the tablets of Step 4 with Opadry.

### Example 2

| Ingredients | % w/w |
|---|---|
| Dapagliflozin Premix | 3.88 |
| Lactose Monohydrate | 42.71 |
| Microcrystalline Cellulose | 23.30 |
| Crospovidone | 11.65 |
| Povidone | 13.6 |
| Isopropyl alcohol | Q.S. |

Extragranular materials

| | |
|---|---|
| Colloidal Silicon Dioxide/Talc | 1.17 |
| Magnesium Stearate/Calcium | 0.78 |
| Stearate/Glyceryl behenate | |

Film coating

| | |
|---|---|
| Opadry coating | 2.91 |

### Procedure:

1. Sift Dapagliflozin premix and Lactose Monohydrate.
2. Co-sift Microcrystalline Cellulose, Crospovidone and Povidone.
3. Load Step 1 & 2 into blender and mix.
4. Granulate step 3 by using binder solution and dry the granules.
5. Lubricate the granules of step 4 and compress into tablets.
6. Coat the tablets of Step 5 with Opadry.

### Example 3

| Ingredients | % w/w |
|---|---|
| Dapagliflozin Premix | 3.85 |
| Lactose | 48.15 |
| Microcrystalline Cellulose | 19.26 |
| Pregelatinized starch | 7.71 |
| Crospovidone | 5.78 |
| Colloidal Silicon Dioxide | 0.58 |
| Magnesium Stearate | 0.39 |

Extragranular materials

| | |
|---|---|
| Microcrystalline Cellulose | 4.62 |
| Crospovidone | 5.78 |
| Colloidal Silicon Dioxide | 0.58 |
| Magnesium Stearate | 0.39 |

Film coating

| | |
|---|---|
| Opadry coating | 2.91 |

### Procedure:

1. Sift Dapagliflozin Premix and Lactose.
2. Co-sift Microcrystalline Cellulose, Crospovidone, Colloidal Silicon Dioxide and Pregelatinized starch.
3. Load Step 1 & 2 into blender and blend.
4. Lubricate blend of step 3 and compact the powder blend.
5. Mill the step 4 compacts.
6. Sift Microcrystalline Cellulose, Crospovidone, and Colloidal Silicon Dioxide
7. Load step 5 & 6 in blender and blend.
8. Lubricate the blend of step 7 and compress into tablets.
9. Coat the tablets of Step 8 with Opadry.

### Example 4

| Ingredients | % w/w |
|---|---|
| Dapagliflozin Premix | 6.28 |
| Lactose Anhydrous | 52.37 |
| Microcrystalline Cellulose | 27.18 |
| Crospovidone | 4.85 |
| Povidone | 3.88 |

Extragranular materials

| | |
|---|---|
| Isopropyl alcohol | qs |
| Colloidal Silicon Dioxide | 0.58 |
| Sodium Stearyl Fumarate | 1.94 |

Film coating

| | |
|---|---|
| Opadry coating | 2.91 |
| Purified water | qs |

### Procedure:

1. Sift Dapagliflozin premix and Lactose anhydrous, Microcrystalline Cellulose, and Crospovidone.
2. Load Step 1 into rapid mixer granulator and dry mix.
3. Dissolve Povidone into Isopropyl alcohol under stirring.
4. Granulate step 2 by using step 3 binder solution.
5. Dry the granules by using rapid dryer.
6. Sift the dried granules and colloidal silicon dioxide.
7. Load step 5 granules and step 6 in blender and blend.
8. Sift Sodium Stearyl fumarate, lubricate the blend of step 7 and compress into tablets.
9. Coat the tablet with Opadry.

### Stability Study—

The formulation prepared according to the example 4 is subjected to stability studies at accelerated conditions of temperature and humidity of 40° C. and 75% RH. Results of these stability studies are summarized in the table 1.

| Related Substances | Initial | 1M | 3M |
|---|---|---|---|
| Impurity-1 | ND | 0.02 | BQL |
| Impurity-2 | ND | ND | ND |
| Impurity-3 | ND | ND | ND |
| Impurity-4 | ND | ND | ND |
| Impurity-5 | BQL | ND | ND |
| Impurity-6 | ND | ND | 0.02 |
| Impurity-7 | ND | ND | ND |
| Impurity-8 | ND | ND | ND |
| Impurity-9 | ND | ND | ND |
| Impurity-10 | ND | ND | ND |
| Highest Unspecified | 0.02 (0.44) | BQL | BQL |
| impurity | | | |
| Total Impurities | 0.10 | 0.02 | 0.02 |
| Total | 0.10 | 0.00 | 0.00 |
| Impurities (Excluding | | | |
| Process impurity) | | | |

ND = Not Detected,
BQL below Quantitation Limit

1. A pharmaceutical composition comprising premix of dapagliflozin with at least one pharmaceutically acceptable excipient(s).

2. The pharmaceutical compositions of claim 1 comprising premix of dapagliflozin with lactose.

3. The pharmaceutical compositions of claim 1 wherein the weight ratio of dapagliflozin to the lactose is from about 1:0.01 to 1:100.

4. The pharmaceutical composition of claim 1 wherein the pharmaceutical composition may be selected from group of powders, granules, pellets, single layered tablets, multilayered tablets, immediate release tablets, sustained release tablet, extended release tablet, modified release tablets.

5. The pharmaceutical composition of comprising premix of dapagliflozin with at least one pharmaceutically acceptable excipient(s) and DPP-IV inhibitor or Biguinide.

6. A method for treating Type II diabetes, comprising, and administering patient in need of such treatment a therapeutically effective amount of pharmaceutical composition of claim 1 comprising premix of dapagliflozin.

7. A method for treating Type II diabetes, comprising, administering patient in need of such treatment a therapeutically effective amount of pharmaceutical composition of claim 5 comprising premix of dapagliflozin.

8. The process for the preparation of pharmaceutical composition of claim 1 comprising following steps;
a. Mixing of premix of dapagliflozin with pharmaceutically acceptable excipient(s)
b. Dry granulation/wet granulation/Direct compression.
c. Film coating of composition

9. The pharmaceutical compositions of claim 1 wherein lactose content of the said formulation ranges about 50% w/w with respect to total weight of formulation.

10. The pharmaceutical compositions of claim 1 wherein lactose content of the premix ranges from about 30-80% w/w with respect to total weight of dapagliflozin lactose premix.
